(19) United States
(12) Patent Application Publication
(10) Pub. No.: US 2009/0326408 A1
(43) Pub. Date: Dec. 31, 2009

(54) Providing Impedance Plethysmography Electrodes

(76) Inventor: Loell Boyce Moon, Ham Lake, MN (US)

Correspondence Address: FISH & RICHARDSON P.C., P.O. BOX 1022, MINNEAPOLIS, MN, 55440-1022, US

(21) Appl. No.: 12/165,430

(22) Filed: Jun. 30, 2008

(51) Int. Cl. A61B 5/053

(52) U.S. Cl. 600/547

(57) ABSTRACT

A method of measuring lung impedance of a subject can include positioning current-injection electrodes on or within the subject in a configuration such that a current injected between the current-injection electrodes propagates substantially through a first lung of the subject, and not through a heart and a second lung of the subject; positioning voltage-measurement electrodes on or within the subject in a configuration such that voltage measuring fields propagate substantially through the first lung and the second lung of the subject, but not through the heart of the subject; and injecting a current between the current-injection electrodes, as positioned, and measuring a resulting voltage between the voltage-measurement electrodes, as positioned, to obtain an impedance measure across lung tissue. The method can further include injecting current and measuring the resulting voltage multiple times over a time period to monitor respiration of the subject over the time period.

### RELATED APPLICATIONS

This application is related to and incorporates by reference application Ser. No. 11/933,872, filed Nov. 1, 2007, by Moon et al.

### BACKGROUND

Animal testing is a critical component of preclinical testing of new pharmaceutical compounds that ultimately may be approved for therapeutic use by human patients. In particular, animal testing can be used to initially assess pharmacodynamics, pharmacokinetics and toxicity of a compound. Based on the animal testing, some compounds may be tested in human clinical trials.

To initially assess pharmacodynamics, pharmacokinetics and toxicity of a compound, the compound may be administered in a controlled manner to laboratory animals (e.g., test subjects, such as mice, rats, guinea pigs, dogs, etc.), and the laboratory animals can be subsequently monitored. Of the various physiological parameters that are frequently monitored during testing, several parameters may be particularly important. For example, the International Conference on Harmonization of Technical Requirements for Registration of Pharmaceuticals for Human Use (ICH)—a group that brings together regulatory authorities in the United States, Europe and Japan for the purpose of harmonizing regulatory guidelines for testing and approving new pharmaceutical compounds—has identified cardiovascular, respiratory and central nervous systems as particularly important. Specifically, the ICH, in its S7A Safety Pharmacology Studies for Human Pharmaceuticals guidelines, has included cardiovascular, respiratory and central nervous systems in a core battery that should be evaluated prior to the first administration of a pharmaceutical substance in humans.

To evaluate the likely effect of a pharmaceutical compound on cardiovascular, respiratory and central nervous systems of humans, the pharmaceutical compound may be tested in various animal models, and various physiological parameters of the animals models may be monitored during the testing. For example, an electrocardiogram (ECG) signal, blood pressure and blood flow rate can be monitored to evaluate the effect of a compound on the cardiovascular system. As another example, motor activity can be monitored (e.g., with electromyography (EMG) parameters), changes in behavior or coordination can be noted, sensory and motor reflex responses can be tracked (e.g., with electroencephalography (EEG) parameters, EMG parameters, or electrooculography (EOG) parameters), and internal body temperature can be monitored to evaluate the effect of a compound on the central nervous system. As another example, respiratory flow, tidal volume, hemoglobin oxygen saturation, and other respiratory parameters can be monitored to evaluate the effect of a compound on the respiratory system.

Various devices can be employed to monitor respiration parameters. For example, a plethysmography chamber can be used to measure respiratory flow of a restrained test subject, such as a laboratory rat, over a period of an hour or two. In some such chambers, the test subject is restrained at the neck and fitted with a hood that is configured with a precise airflow monitoring system. In other chambers, animals are permitted a small amount of movement within a small enclosure that is also configured with a precise airflow monitoring system. Respiration parameters can also be obtained from anesthetized animals with a breathing tube fitted with precise pressure or flow sensors. In addition, jacket-based systems can allow certain respiration parameters to be gathered from cooperative animals over a period of one or two days.

### SUMMARY

During preclinical testing of pharmaceutical compounds on test subjects (or in other research studies of the effect of other test substances on test subjects), various physiological parameters of the test subjects can be monitored with a wireless implantable device. The wireless implantable device can facilitate collection of physiological data from unrestrained and unanesthetized test subjects. In particular, respiration parameters can be obtained in a minimally invasive manner, using subcutaneously implanted electrodes. More specifically, time-varying thoracic impedance values can be obtained, from which tidal volume, respiratory rate, inspiratory time or interval and flow, and expiratory time or interval and flow can be determined. Certain electrode configurations can be particularly effective for obtaining respiration parameters. For example, four-electrode configurations in which electrodes are disposed on at least three different lead wires can, when the lead wires are appropriately placed, facilitate measurement of signals that include a large respiration component and small non-respiration components, such as signals related to cardiac activity or muscle movement.

In some implementations, a method of measuring lung impedance of a subject includes positioning current-injection electrodes on or within the subject in a configuration such that a current injected between the current-injection electrodes propagates substantially through a first lung of the subject, and not through a heart and a second lung of the subject; positioning voltage-measurement electrodes on or within the subject in a configuration such that voltage measuring fields propagate substantially through the first lung and the second lung of the subject, but not through the heart of the subject; and injecting a current between the current-injection electrodes, as positioned, and measuring a resulting voltage between the voltage-measurement electrodes, as positioned, to obtain an impedance measure across lung tissue. The method can further include injecting current and measuring the resulting voltage multiple times over a time period to monitor respiration of the subject over the time period.

The current-injection electrodes can include a first electrode positioned laterally on a right side of the subject, and a second electrode positioned mid-laterally on the right side of the subject. The voltage-measurement electrodes can include a third electrode positioned in a pectoral region of the subject, and a fourth electrode positioned mid-laterally. In some implementations, the current-injection electrodes and the voltage-measurement electrodes are implanted within the subject. In some implementations, the current-injection electrodes and the voltage-measurement electrodes are surface electrodes placed on a surface of the subject. In some implementations, the first electrode is positioned near an axilla. In some implementations, the second electrode is positioned near an intersection of a rib cage and an abdomen of the subject. In some implementations, the fourth electrode is positioned on a left front side of a thorax of the subject.

In some implementations, a method of measuring a respiration parameter in a living being includes implanting in the living being a system, the system comprising a) a wireless transmitter; b) three distinct lead wires; and c) four distinct electrodes disposed on the three distinct lead wires, wherein each of the three distinct lead wires has disposed thereon at least one of the four distinct electrodes and wherein implanting the three distinct lead wires comprises implanting the three lead wires subcutaneously or sub-muscularly; injecting a current between two of the four distinct electrodes to create a current field in the living being, and measuring a resulting voltage between the other two of four distinct electrodes; transmitting from the wireless transmitter to a receiver that is external to the living being a value corresponding to the measured voltage; and determining from the value a respiration parameter for the living being.

The living being can have an abdomen, a thorax generally bounded by a rib cage, and an axilla; and implanting the system can include implanting the three distinct lead wires such that a) a first of the four electrodes is positioned on a right side of the thorax, mid-laterally near an intersection of the rib cage and the abdomen; b) a second of the four electrodes is positioned on the right side of the thorax near the axilla; c) a third of the four electrodes is implanted medially in a left or right pectoral region; and d) a fourth of the four electrodes is implanted mid-laterally on a left ventral side of the thorax.

In some implementations, injecting the current includes injecting the current between the first and second electrodes, and measuring the voltage comprises measuring the voltage between the third and fourth electrodes. In some implementations, injecting the current includes injecting the current between the third and fourth electrodes, and measuring the voltage comprises measuring the voltage between the first and second electrodes. Implanting the three distinct lead wires can include implanting the three distinct lead wires such that the third electrode is disposed cranially relative to the fourth electrode. In some implementations, the system includes four distinct lead wires, and one of the four distinct electrodes is disposed on each of the four distinct lead wires. In some implementations, implanting the system includes subcutaneously implanting the three distinct lead wires such that a) the current field extends into a thorax of the living being in a manner that intersects lung structures of the living being, and b) the voltage is measured form a voltage field that intersects the lung structures but does not substantially intersect heart structures of the living being.

In some implementations, a method of measuring a respiration parameter in a living being includes implanting in the living being a system, the system comprising a) a wireless transmitter; b) three distinct lead wires; and c) four distinct electrodes disposed on the three distinct lead wires, wherein each of the three distinct lead wires has disposed thereon at least one of the four distinct electrodes and wherein implanting the three distinct lead wires comprises implanting the three lead wires subcutaneously or sub-muscularly; developing a voltage potential between two of the four distinct electrodes to create a voltage field in the living being, and measuring a resulting current between the other two of four distinct electrodes; transmitting from the wireless transmitter to a receiver that is external to the living being a value corresponding to the measured current; and determining from the value a respiration parameter for the living being.

In some implementations, a method of measuring lung impedance of a subject includes positioning current-injection electrodes within the subject in a configuration such that a current injected between the current-injection electrodes propagates as a plurality of current fields through at least one lung of the subject; positioning voltage-measurement electrodes within the subject in a configuration such that a) corresponding voltage-measuring fields intersect the plurality of current fields substantially in the at least one lung and b) the voltage-measuring fields do not substantially intersect the plurality of current fields in a heart of the subject; and injecting a current between the current-injection electrodes, as positioned, and measuring a resulting voltage between the voltage-measurement electrodes, as positioned, to obtain an impedance measure across tissue of the at least one lung.

The details of one or more implementations are set forth in the accompanying drawings and the description below. Other features, objects, and advantages will be apparent from the description and drawings, and from the claims.

### DESCRIPTION OF DRAWINGS

FIG. 1 is a diagram of an example environment in which an implantable monitoring device may be used.

FIG. 2A is a block diagram of an example implantable monitoring device.

FIGS. 2B and 2C are block and schematic diagrams, respectively, of an impedance sensor that can be included in an implantable monitoring device.

FIGS. 2D-2I illustrates various example configurations of lead wires that can be used to monitor impedance.

FIG. 3A is an illustration depicting how the device shown in FIG. 2 may be implanted in a laboratory animal.

FIGS. 3B and 3C illustrate example electrode configurations for the device shown in FIG. 3A.

FIG. 3D provides an anatomical reference for the examples of FIGS. 3B and 3C.

FIGS. 3E and 3F illustrate example signals that can be obtained from the configurations of FIGS. 3B and 3C, respectively.

Like reference symbols in the various drawings indicate like elements.

### DETAILED DESCRIPTION

During preclinical testing of pharmaceutical compounds on test subjects (or in other research studies of the effect of other test substances on test subjects), various physiological parameters of the test subjects can be monitored with a wireless implantable device. The wireless implantable device can facilitate collection of physiological data from unrestrained and unanesthetized test subjects. In particular, respiration parameters can be obtained in a minimally invasive manner, using subcutaneously implanted electrodes. More specifically, time-varying thoracic impedance values can be obtained, from which tidal volume, respiratory rate, inspiratory time or interval and flow, and expiratory time or interval and flow can be determined. Certain electrode configurations can be particularly effective for obtaining respiration parameters. For example, four-electrode configurations in which electrodes are disposed on at least three different lead wires can, when the lead wires are appropriately placed, facilitate measurement of signals that include a large respiration component and small non-respiration components, such as signals related to cardiac activity or muscle movement.

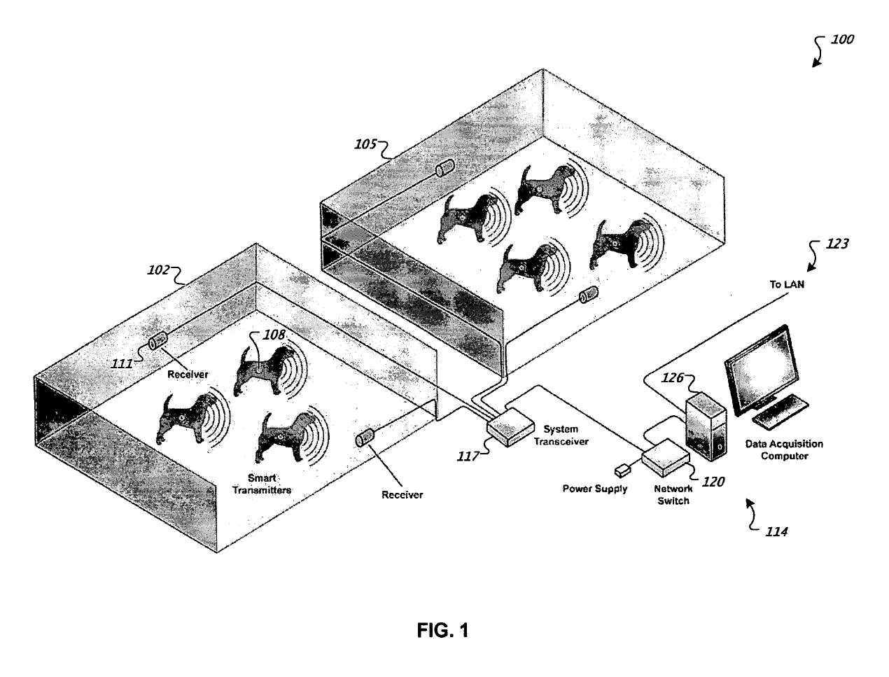

FIG. 1 illustrates one example environment 100 in which physiological parameters of test subjects (e.g., laboratory animals) can be captured with an implanted device in a controlled environment (e.g., in the context of preclinical testing of pharmaceutical components). In FIG. 1, the test subjects depicted are dogs; however, the environment 100 can be used to monitor physiological parameters of any kind of laboratory animal. As shown in one implementation, the environment 100 includes containment areas 102 and 105. Each containment area 102 or 105 can be configured to house multiple animals, as shown (e.g., to permit natural social interaction between the animals). Alternatively, containment areas can be configured to house a single animal. In other implementations, the areas 102 or 105 could simply be regions in which unrestrained test subjects, such as human patients, are within range of the below-described monitoring equipment.

As depicted in one implementation, a monitoring device, such as the monitoring device 108, is implanted in each animal. The monitoring device 108 (or implantable device 108) can include one or more sensors configured to capture one or more physiological parameters of the animal, and a transmitter configured to transmit captured physiological parameters to a receiver, such as the receiver 111 (which, in some implementations, may be replaced by a transceiver). As shown, various receivers are located in the containment areas. Each receiver is connected to an acquisition system 114, which can receive, store and analyze physiological data. In one implementation, as shown, various receivers are connected to a system transceiver 117, which can combine data received from multiple receivers into a single data stream (or smaller number of data streams). The acquisition system 114 can include network connections, such as a network switch 120 or LAN connection 123, to permit the system to monitor a larger number of containment areas or to facilitate remote access of data. The acquisition system 114 can include a storage and analysis device, such as a computer 126, which can be used to receive, store, display and analyze captured physiological data.

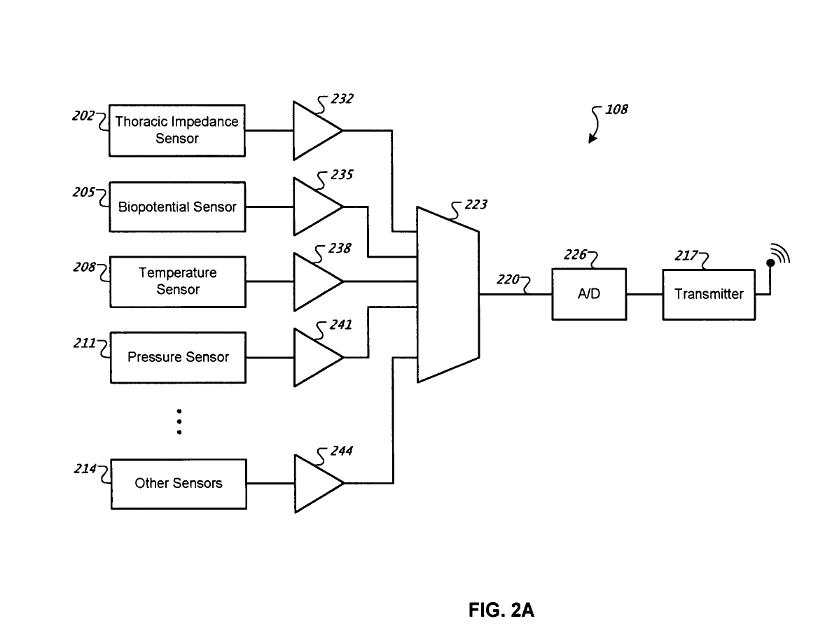

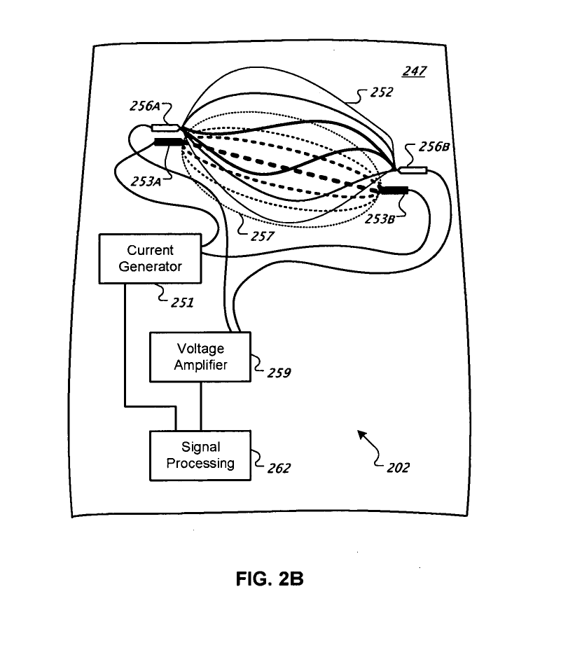

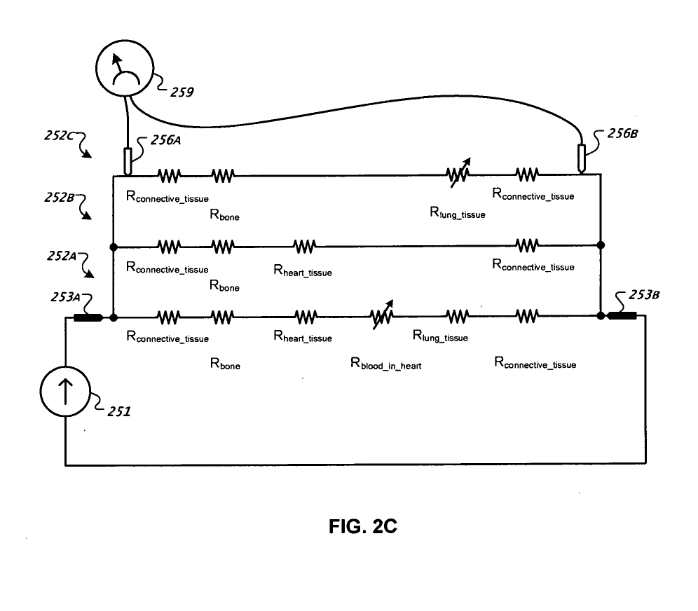

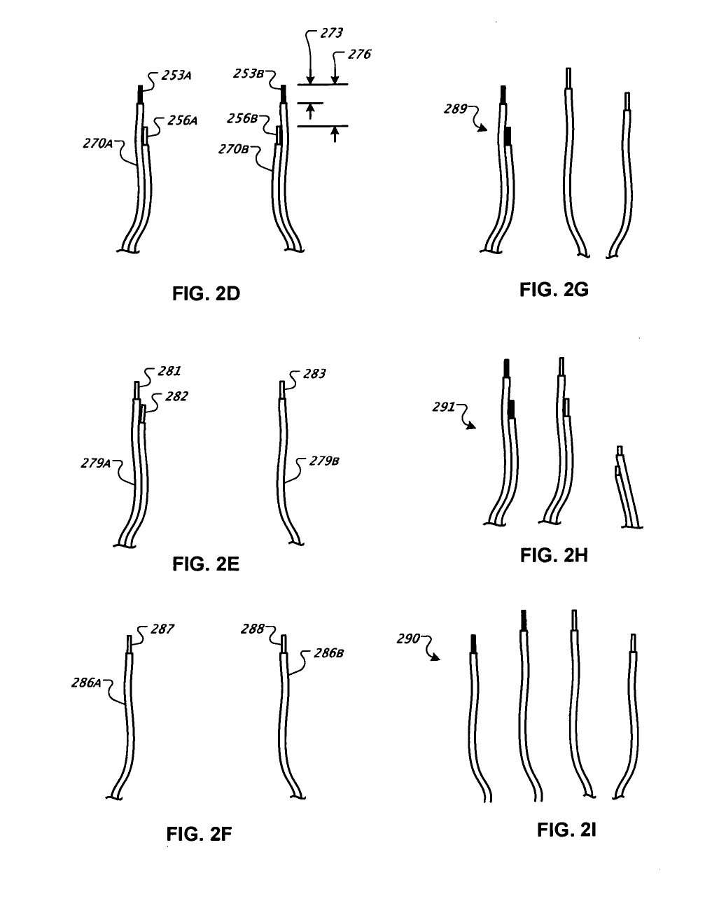

FIG. 2A illustrates additional details of the example implantable device 108 that is depicted in FIG. 1. As described above, the implantable device 108 can include a number of sensor devices that can be implanted in a test subject. The sensor devices can include, for example, a thoracic impedance sensor 202, which is described in greater detail below; a biopotential sensor 205 (e.g., a sensor for measuring biopotential signals, such as electroencephalography (EEG) signals, electrocardiogram (ECG) signals, electromyography (EMG) signals, or electrooculography (EOG) signals); a temperature sensor 208; a pressure sensor 211 (e.g., for sensing blood pressure or pressure of an internal cavity); and other sensors 214.

Other sensors 214 can include, for example, an accelerometer, which can be used to detect position, movement or behavior of a test subject. Any other sensor configured to monitor a physiological parameter of a test subject can be included in the implantable device 108. In particular, some implantable devices 108 include a suite of sensors that enable researchers to obtain a large amount of data (e.g., data that is typically collected during pharmaceutical testing) with a single device. For example, a suite of sensors could include one or more of the following: blood flow sensors, edema sensors, ionic state sensors (e.g., for sensing K⁺, NA^{+ , CA}⁺), gas sensors (e.g., for sensing NO, O, O₂, or CO₂), pH sensors, glucose sensors, insulin sensors, oxygen saturation sensors, various pressure sensors, posture and activity sensors, sound sensors (e.g., for heart sound or rales detection), etc.

Values of physiological parameters captured by the various sensors 202-214 can be transmitted by a transmitter 217 to an external system, such as the receiver 111 and acquisition system 114 (external system 114) shown in FIG. 1. As shown in one implementation, values of the physiological parameters can be multiplexed into a single signal 220 with a signal combiner 223 (e.g., a multiplexer), and the signal 220 can be converted from an analog format to a digital format with an analog-to-digital converter 226 (A/D 226) before being transmitted.

In some implementations, signals from various sensors can be amplified, or the signals can otherwise be processed (e.g., with amplifiers 232-244). Gain may be individually configurable for each sensor 202-214, and in some implementations, filtering may be applied to individual or multiple signals. For example, the amplifier 235 may include a filter (not explicitly shown) to filter ECG signals captured by the biopotential sensor 205 out of the signal that is captured by the thoracic impedance sensor 202.

The A/D function is shown for purposes of example as following the multiplexer 223, but in some implementations, signals are digitized before being multiplexed. In other implementations, signals may be transmitted in analog form (e.g., encoded in a form that permits an analog representation (e.g., analog frequency modulation, amplitude modulation, pulse width modulation, pulse position modulation, etc.)).

In some implementations, each sensor signal is allotted a timeslot, such that the resulting signal 220 is a time-division multiplexed signal. For example, the signal 220 could be formatted into frames with a number of timeslots, and each sensor could provide data for a particular timeslot in each frames.

FIGS. 2B and 2C are a block diagram and a schematic diagram, respectively, illustrating additional details of the example thoracic impedance sensor 202 in one example configuration. The thoracic impedance sensor 202 can be used to measure thoracic impedance in body tissue 247 of a test subject. In operation, the thoracic impedance sensor 202 can detect changes in thoracic impedance resulting from physiological changes. Bone, organ tissue (e.g., tissue of the heart and lungs) and connective tissue present a relatively constant impedance (as depicted by the fixed resistances in the schematic diagram shown in FIG. 2C); air is highly resistive, and ionized fluids (e.g., blood) have a low resistance. Accordingly, variations in air volume and changes in blood flow can directly cause changes in transthoracic impedance (as indicated by the variable resistances in FIG. 2C). A correlation has been established between changes in thoracic impedance during respiration cycles and the tidal volume of air inhaled and exhaled during the respiration cycles. Correlations have also been established between cardiac stroke output and certain thoracic impedance changes. Longer-term correlations have been established between baseline thoracic impedance and fluid volume in tissue or organs. For example, a worsening condition of edema may be detected through monitoring and analysis of changes in baseline thoracic impedance changes over relatively longer periods of time (e.g., changes over hours, days or weeks, rather than changes from breath-to-breath or heart beat-to-heart beat). Accordingly, various physiological parameters can be derived from measurements of thoracic impedance.

In one implementation as shown, the thoracic impedance sensor 202 includes a current generator 251 that generates a current signal, which passes through body tissue 247 of the test subject from an electrode 253A to an electrode 253B. The current signal passes between the electrodes along many different paths (as represented by the different current paths in FIG. 2C) and through different body tissues and structures. The amplitude of the signal is modulated by changes in thoracic impedance, which in many implementations, results from changes in air volume in the lungs and blood volume in the heart. The modulation of the current signal can be detected as a change in potential difference between different points in the body tissue 247. Put another way, a time-varying voltage can be detected in the body tissue 247, and the magnitude of the time-varying voltage is related to the magnitude of the original current signal, the base thoracic impedance, and the change in thoracic impedance caused by respiration and other physiological processes (e.g., blood flow variations related to cardiac function). In one implementations as shown, a separate set of electrodes 256A and 256B and a voltage amplifier 259 (e.g., one or more field effect transistors (FETs) and a differential amplifier, in one implementation) can detect the voltage difference created by the current signal and the impedance of the body tissue 247 along a path between the voltage electrodes 256A and 256B. In other implementations, the same electrodes may be employed to both provide the current signal and detect the corresponding voltage signal. Other specific example electrode and lead wire configurations are described in more detail below, with reference to FIGS. 2D-2I.

The above-described current and voltage signals can be graphically depicted as lead fields between corresponding electrodes. For example, as shown in FIG. 2B, a first lead field 252 can correspond to and graphically represent a current signal. A second lead field 257 can correspond to and graphically represent voltages that are induced in body tissue by the current signal (the first lead field 252). By graphically depicting the current and voltage signals as lead fields, one may visualize physiological components (e.g., respiration components, cardiac components, components associated with fluid retained in body tissue, muscle-movement components, etc.)) of the voltage signal (e.g., the second lead field 257), for example, based on the graphical relationship of the voltage lead field 257 to the current lead field 252. Changes in impedance of organs or tissue that are disposed within regions where the lead fields intersect will generally be the strongest contributors to changes in the voltage signal, since tissue or organs in such regions receive a strong current signal and are located within a region in which the voltage leads are particularly sensitive. Conversely, changes in impedance of organs or tissue disposed at locations where the lead fields do not intersect will contribute little to changes in the voltage signal, since such organs or tissue are either located outside a region in which the voltage leads are most sensitive, or since such organs or tissue receive little, if any, of the current from the current leads.

As a reader who is familiar with the present art will appreciate, the above description is, for purposes of explanation, a simplification of the mode of propagation of fields within a body. The actual lead fields will be influenced by the impedance of various internal structures and fluids through which the lead fields travel. Each structure or fluid may have a different impedance, and as those structures and fluids move within the body, the impedance, and thus the lead fields, may dynamically change. With respect to the current field, currents injected between the two current electrodes will generally follow paths of least resistance, primarily through fluids and tissue that are least resistive. Moreover, the current will generally follow multiple parallel paths, and those paths may change dynamically, as body structures and fluids move an dynamically change the internal impedance of the body. For example, current flowing over or through a lung may traverse a greater amount of tissue as the lung expands during respiration, resulting in a greater voltage change over or through that tissue when the lung is in its most expanded state. Current paths that are farther away from the electrodes (e.g., longer current paths, or those current paths having a greater “radius,” as depicted in FIG. 2B) may generally conduct less current than current paths that are closer to the electrodes and that have shorter paths. This variation is the amount of current conducted is depicted by the variation in thickness of the lines depicting the current field. This variation in current field is further illustrated and described with reference to the parallel current paths 252A, 252B and 252C shown in the schematic shown in FIG. 2C. The lead fields corresponding to the voltage electrodes (e.g., those paths along which the voltage probes are most sensitive) may also take paths that are influenced by distances from the electrodes and from the specific organs and body fluids (and more particularly, their corresponding impedances) disposed between the electrodes.

In general, one may expect thoracic impedance measurements to correspond most to regions in which the current and voltage fields intersect. Accordingly, the voltage measurements will generally correspond most to those regions where the voltage field intersects the current field at points of greatest magnitude, and be influenced relatively less by other current paths carrying smaller currents (e.g., current paths represented by thinner lead lines). Thus, by carefully positioning electrodes to influence the location of current and voltage lead field, one can focus thoracic impedance measurements more on specific regions than on other regions (e.g., more on a lobe of a lung and less on the heart, to, for example, obtain a respiration signal that is relatively free of cardiac artifacts).

The schematic diagram shown in FIG. 2C can further clarify the effect of electrode placement on current and voltage signals. In particular, placement of the current electrodes (sometimes referred to as excitation electrodes) can determine which body structures, fluids and tissue the current signal flows through. The example in FIG. 2C illustrates a circuit in which the current signal flows, in various current paths 252A, 252B and 252C, through connective tissue near one current electrode (electrode 253A), bone, heart tissue, blood in the heart, lung tissue, air in the lungs, and connective tissue near a second current electrode (electrode 253B).

By repositioning the current electrodes 253A and 253B, it may be possible to direct current through a portion of the lungs in a manner that prevents most of the current from passing through the heart. Put another way, given that the magnitude of the various current fields diminishes in predictable ways as those fields extend farther from the electrodes and pass through different organs, tissue and body fluids, one can position the electrodes to favor one of the example current paths 252A-C over the others. More particularly, to obtain a thoracic impedance measurement that favors respiration over cardiac function, one can place the electrodes 253A and 253B in a manner that favors current through current path 252C, over current path 252A. In such a configuration, the voltage leads 256A and 256B may still detect some voltage signal associated with the heart tissue or blood, since the heart tissue and blood are still within the region of sensitivity of the voltage electrodes (e.g., within the lead field of the voltage electrodes); however, this signal may not be as strongly correlated with the current signal than it would be if the current path 252A were favored (by the placement of the electrodes 253A and 253B) over current path 252C, since not as much current passes through the heart tissue when current path 252C is favored.

Placement of the voltage electrodes 256A and 256B can also affect the corresponding voltage signal, and thus any impedance calculation that is derived from the voltage signal. With continued reference to FIG. 2C, changing the position of the voltage electrodes 256A can cause the lead field of the electrodes 256A and 256B to be focused in particular regions, such as on lung tissue between the electrodes. In such an example configuration, even though the current signal may continue to flow through other connective tissue, bone structures and the heart, the voltage electrodes 256A and 256B may not be as sensitive to the voltage drop across these structures (caused by their corresponding impedances), since these structures are primarily outside of the lead field of the voltage electrodes.

As the above discussion indicates, one may be able to place electrodes in positions that are optimal for measuring particular physiological parameters (e.g., cardiac parameters, respiration parameters, pathological conditions, etc.) by considering how placement of electrodes will affect corresponding lead fields. In some test subjects, and in some regions of a test subject's body, consideration of the lead fields may require extensive consideration of physiological and electrical properties of the tissue and organs. That is, because of different base impedance values of different tissue, organs and bodily fluids, the lead fields that couple two electrodes may not resemble a spherical pattern that one might expect if the electrodes were placed in a uniform material. Accordingly, in some implementations, determining lead fields for particular test subjects may require extensive simulation or empirical study.

Optimally placing electrodes can include placing electrodes in such a way that certain physiological components are excluded or minimized from measurement. For example, for studies that focus on respiration, it may be advantageous to position electrodes to maximize the contribution to the voltage signal of variations in impedance of a test subject's lungs, and to minimize the contribution of variations in impedance of a test subject's heart. Likewise, for studies that focus on cardiac output, it may be advantageous to position electrodes to minimize respiration components while maximizing the cardiac components. Positioning electrodes to optimally obtain particular signals is further discussed below.

In some implementations, a separate signal processing element 262 converts the detected voltage to an impedance (e.g., by dividing the magnitude of the detected voltage by the magnitude of the current signal). In other implementations, the voltage signal is maintained as such, and the conversion to an impedance value can be performed elsewhere in the system (e.g., in the external system 114). Other signal processing may be performed by the signal processing element 262. For example, the signal processing element 262 may filter the signal (e.g., to remove noise at a particular frequency or range of frequencies; more particularly, some implementations employ a band-pass filter having a center frequency at the frequency of the current signal), or the signal processing element 262 may digitize the detected voltage or calculated impedance value.

The current signal can be any signal that will create a detectable voltage signal without causing other adverse effects (e.g., muscle sensation or stimulation, pain, tissue destruction, etc.). Frequently, the current signal is a very low, periodic current signal. For example, the current signal can be a sinusoidal or pulsed signal having a frequency of 1-100 kHz (e.g., 25 kHz) and an amplitude of 50-400 uA (e.g., 200-300 uA). In some implementations, a pulsed (e.g., square wave) signal may be preferred over other signals because a pulsed signal can be easy to generate and may also require less power than, for example, a sinusoidal signal. Other implementations employ other kinds of signals (e.g., triangle, bi-phasic, etc.), and may employ other frequencies or amplitudes. Moreover, other implementations may reverse the current and voltage signals. That is, in such implementations, a voltage potential may be developed between electrodes, and a corresponding current may be measured.

In some implementations, various parameters may be adjustable or programmable, either manually (e.g., remotely, from signals transmitted from the external system 114 to a receiver and corresponding processing circuitry (not shown) in the implantable device 108). In particular, for example, amplitude or frequency of the current signal generated by the current generator 251 may be adjustable (e.g., to facilitate use of the implantable device 108 in test subjects of various sizes). As another example, the gain for individual sensors (e.g., the gain of amplifiers 232-244) may be adjustable (e.g., manually, or automatically—based on processing circuitry internal to the implantable device 108) to facilitate a high signal-to-noise ratio in a variety of operating environments. In some implementations, frequency and current amplitude are both adjustable to maximize the signal-to-noise ratio while minimizing power consumption.

In FIG. 2B, four discrete electrodes 253A, 253B, 256A and 256B are shown (a tetrapolar lead arrangement). Additional details of such a tetrapolar arrangement, and other arrangements, are shown in and described with reference to FIGS. 2D-2I. In particular, six example electrode and lead arrangements are shown in FIGS. 2D-2I. In the implementation shown in FIG. 2D, the four electrodes 253A, 253B, 256A and 256B are disposed on two lead wires 270A and 270B—two electrodes on each lead wire. In some implementations, for example implementations in which the lead wires 270A and 270B and corresponding electrodes are implanted in small animals (e.g., rodents), the electrodes may have an approximate length 273 of ½ cm (e.g., lead exposure, or electrode length), and a distance 276 of approximately 1 cm may separate multiple electrodes (e.g., electrodes 253B and 256B) on a single lead wire (e.g., lead wire 270B). In other implementations, electrode lengths and distances between electrodes on a single wire can have different dimensions. For example, in larger animals (e.g., canines), electrodes may have an approximate length 273 of 1 cm, and a distance 276 of approximately 5 cm.

In general, the dimensions and placement of the electrodes (e.g., electrode length and electrode separation) can be optimized to capture a good signal, based, for example, on the size of the test subject. In particular, for example, the closer the electrodes 253B and 256B are (e.g., in a tetrapolar configuration), the more artifacts (e.g., from movement) that may be picked up. As electrodes 253B and 256B are separated, the signal may improve. In addition, distance between the electrodes 253B and 256 can control the depth of the impedance measurement (that is, a greater separation can facilitate a more deep impedance measurement in the test subject than a smaller separation). Amplitude of the current signal can also affect signal quality (e.g., signal-to-noise ratio). Thus, amplitude of the current can be increased for larger animals, within constraints imposed, for example, by power consumption requirements and limits on current that may be necessary to prevent tissue from being stimulated.

In many tetrapolar implementations, electrodes on the same lead wire are configured to remain a fixed distance from each other following implantation. In particular, for example, the electrodes 253B and 256B may be rigidly fixed relative to each other to prevent changes in the detected voltage signals once the lead wires 270A and 270B are implanted in the test subject.

The electrodes themselves can be made of any material that is suitable for implantation in a living being. For example, some electrodes are made of bare wire formed from or coated with a gold or titanium alloy. In some implementations, the electrodes are merely exposed portions of the lead wires. In other implementations, the electrodes are separately formed (e.g., to increase their surface area or to provide a custom shape) and attached to corresponding lead wires. Electrodes may be coated to enhance signal pickup, minimize corrosion or chemical or ion interaction with the tissue, or prevent tissue from sticking to or growing onto the electrodes. In particular, for example, some electrodes are coated with polytetrafluoroethylene (PTFE). As another example, some electrodes are coated with platinum black.

The lead wires 270A and 270B are depicted as parallel, two-conductor lead wires, but in other implementations, the lead wires can have different arrangements. In particular, for example, multi-conductor lead wires can have a co-axial or co-radial arrangement. The conductors within various kinds of lead wires can be cylindrical or flat.

Configuration 289, shown in FIG. 2G, illustrates another tetrapolar arrangement in which one lead wire has two electrodes disposed thereon, with two other electrodes being provided on their own lead wires. Separate lead wires for each of one or more electrodes may be advantageous to minimize the size (e.g., diameter) of the lead wire itself, where electrodes may be separated by a greater distance than the distance 276. For example, separate lead wires may be advantageous for larger test subjects, or for test subjects in which electrodes may be placed in distinct regions of the body. Configuration 290, shown in FIG. 2I, illustrates another tetrapolar arrangement in which each electrode is disposed on its own lead wire.

Once implanted, lead wires can be anchored in various manners in a test subject—for example, to control the depth and orientation of the current field. In particular, the lead wires can be directly sutured (e.g., with the aid of tabs) to skin or muscle of the test subject, or the lead wires can be threaded though a sleeve which is itself sutured to the skin or muscle of the test subject. Alternatively, a mesh (e.g., a Dacron™ mesh—not shown in FIG. 2D) can be provided to serve as an anchor surface on the end of a lead wire. Other known anchoring techniques can be employed to prevent a lead wire from moving in an undesirable manner once it is implanted.

Other configurations of lead wires and electrodes are shown in FIGS. 2E-2I. In particular, for example, lead wires 279A and 279B in FIG. 2E illustrate a tripolar arrangement in which two electrodes 281 and 282 are disposed on one lead wire 279A and a third electrode 283 is disposed on the second lead wire 279B. In a tripolar arrangement a current signal can be provided between electrodes 281 and 283, and a voltage signal can be sensed between electrodes 282 and 283. In such an arrangement, the electrode 283 can be common to both the current-generating and voltage-sensing circuits. In a bipolar implementation shown in FIG. 2F, both electrodes 287 and 288 can be common to the current-generating and voltage-sensing circuits, and each electrode can be disposed on its own respective lead wire 286A or 286B.

Other configurations are possible. For example, by employing greater numbers of electrodes, thoracic impedance measurements can be captured from a number of different regions of the test subject's body. Moreover, by employing greater numbers of electrodes, certain electrodes can be employed to optimally measure impedance, while other electrodes can be optimally employed to measure other physiological parameters. A configuration 291, shown in FIG. 2H, illustrates a six-electrode, three-lead wire arrangement. In such an arrangement, four of the electrodes can be employed for measuring impedance (e.g., two electrodes for providing a current signal, and two electrodes for measuring a corresponding voltage signal); the remaining two electrodes may be used for other purposes, such as for separately measuring, for example, an ECG, EMG or EOG signal from a different location than the voltage or current electrodes.

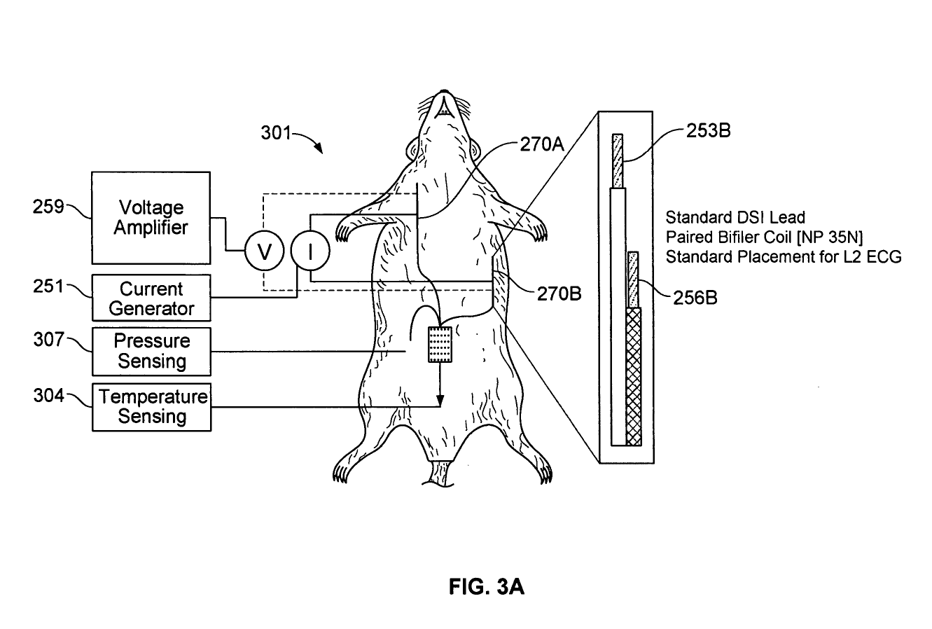

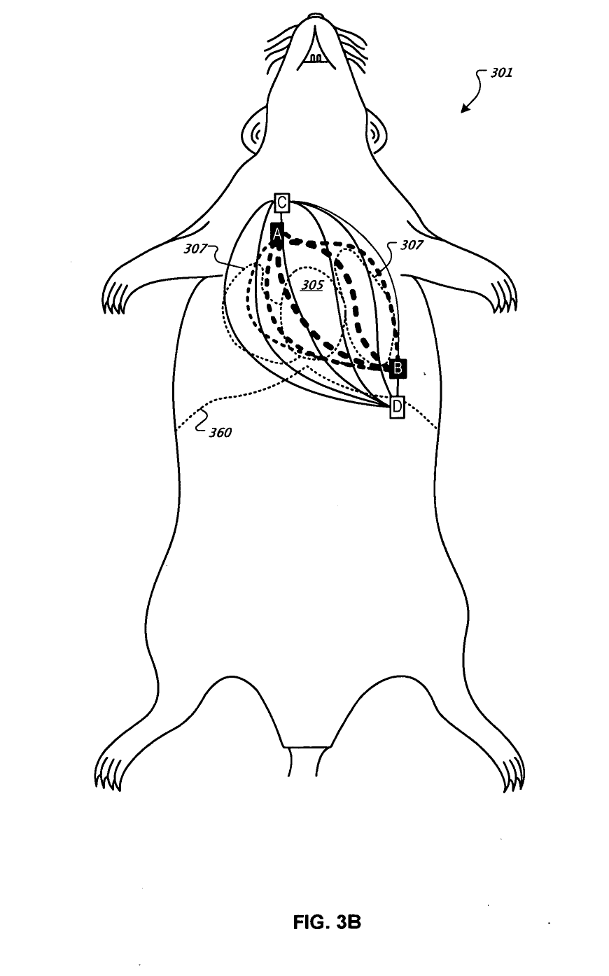

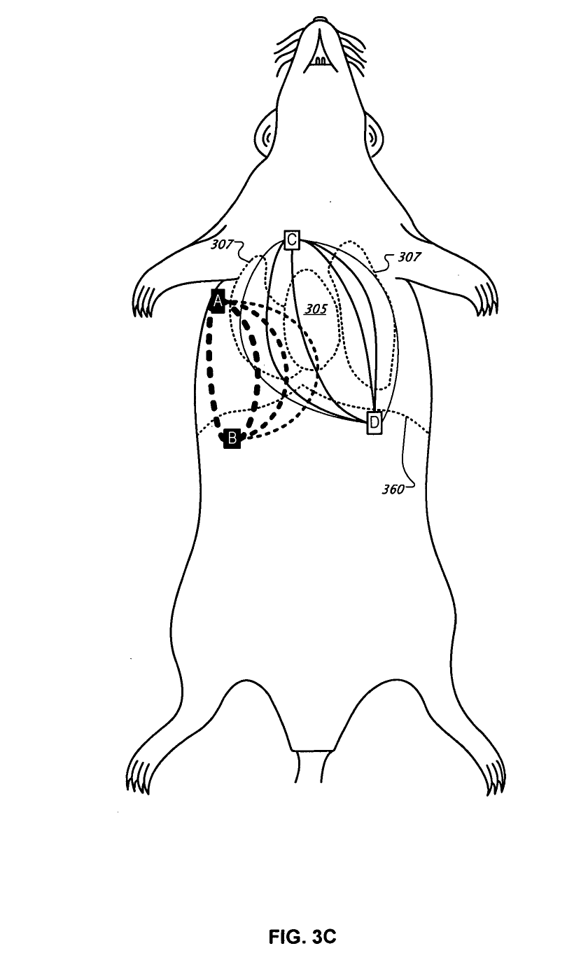

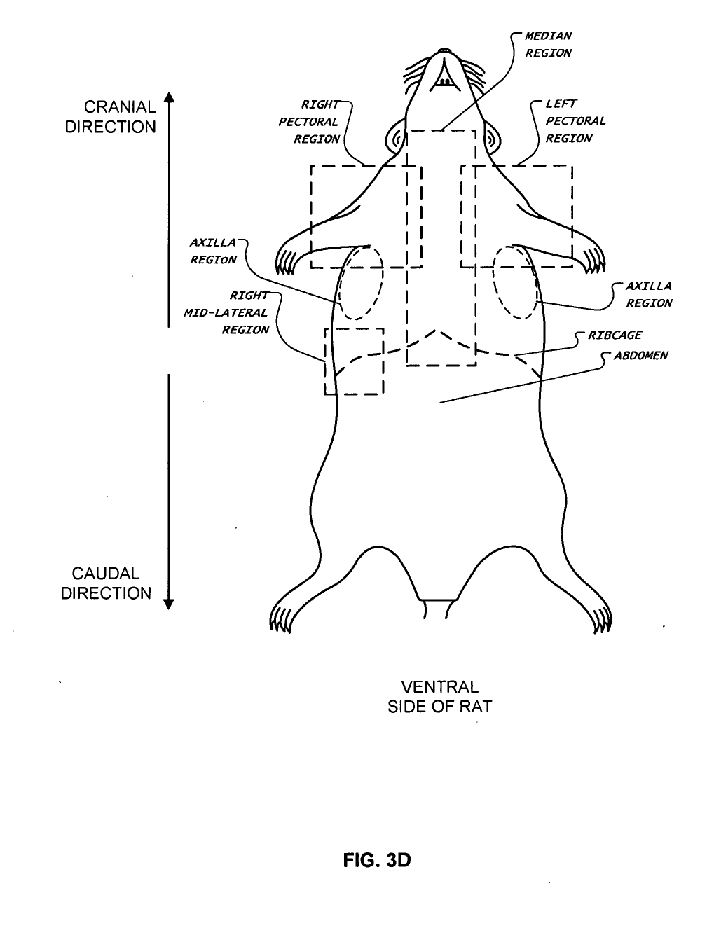

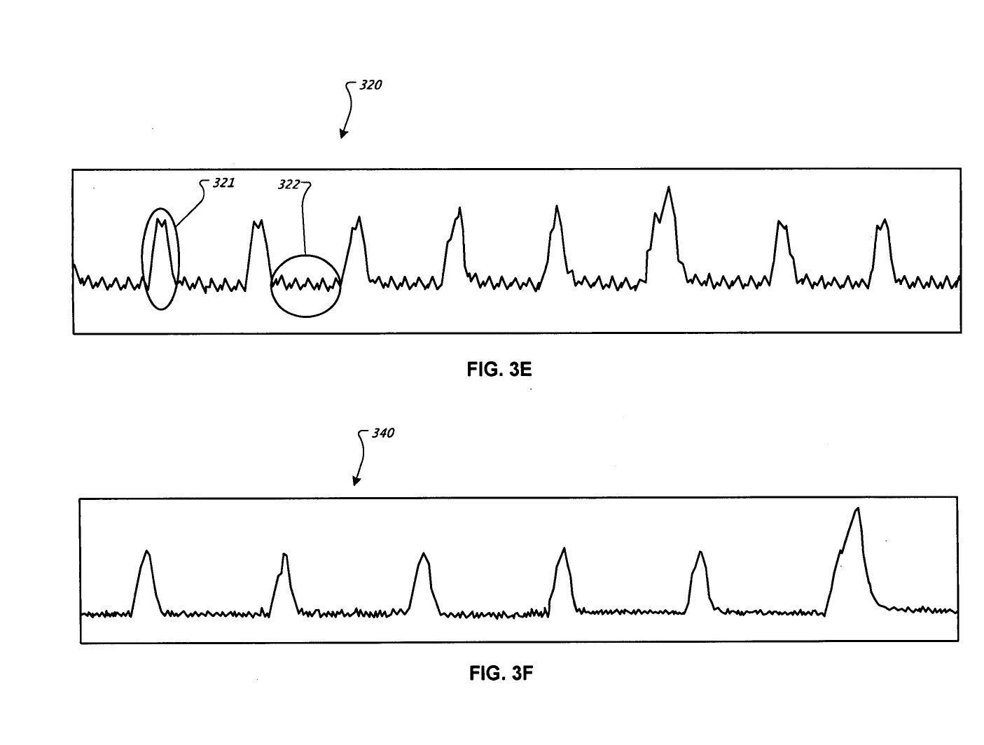

Although described above primarily in the context of measuring thoracic impedance, the lead wires (e.g., lead wires 270A and 270B) for measuring thoracic impedance can also be used to capture other biopotential information. In particular, for example, the electrode 256B (shown in FIG. 2D and FIG. 3) can be used to capture one ECG signal, and the electrode 256A can be used to capture another ECG signal (e.g., another standard channel of single-ended ECG information). Alternatively, the electrodes 256A and 256B can together provide a differential biopotential signal. In some implementations, the biopotential signals are captured at substantially the same time that thoracic impedance values are obtained (e.g., signals on the appropriate electrodes may be sampled at some frequency, and the samples may alternate between sampling thoracic impedance information (e.g., voltage induced by the above-described current signal) and sampling ECG information (e.g., each sample or based on some other pattern, such as one thoracic impedance sample for every five ECG samples). In such implementations, the ECG (or other biopotential information) may be sampled in a manner that is synchronized with the current signal (e.g., such that the sample is made when the current generator is not actively providing current to the body tissue, such as the off portion of a pulsed current signal). In other implementations, the leads may be used for either capturing ECG or other biopotential information, or for capturing thoracic impedance information, and the current function of the leads may be remotely programmable or adjustable. Other configurations and measurements are contemplated. For example, all four electrodes 253A, 253B, 256A and 256B in the tetrapolar configuration shown in FIG. 2D could be employed to capture biopotential information.

FIG. 3A is a diagram of the implantable monitoring device 108, shown implanted in a laboratory rat 301, which can be used to obtain impedance measurements. For purposes of example, a tetrapolar lead arrangement is depicted, and a temperature sensor 304 and pressure sensor 307 are also shown to be included in the device and implanted in the laboratory rat 301. In one implementation as shown, pairs of electrodes are physically disposed in the two lead wires 270A and 270B. In this example, as described with reference to FIGS. 2B and 2C, a current signal is propagated from a first electrode 253A (not labeled in FIG. 3) on a first lead wire 270A to a second electrode 253B on a second lead wire 270B, and a resulting voltage difference is detected between a third electrode 256A (not labeled in FIG. 3) on the first lead wire 270A and a fourth electrode 256B on the second lead wire 270B. The current generator 251 and voltage amplifier 259 are depicted outside of the laboratory rat 301 for clarity, but the reader will appreciate, in light of the above description with reference to FIGS. 1 and 2A-2D, that the current generator 251, voltage amplifier 259 and other components can be fully implanted in the laboratory rat 301.

Placement of the lead wires and corresponding electrodes can impact the quality of the sensed thoracic impedance. For example, systems in which the voltage electrodes 256A and 256B are placed in a manner that causes the voltage signal to include a cardiac component may require digital filtering circuitry to separate cardiac and respiration components (e.g., in order to obtain a clean respiration component). The placement of the electrodes as shown in FIG. 3A, and a corresponding voltage signal that may be obtained by such a placement is further described with reference to FIG. 3B.

FIG. 3B depicts laboratory rat 301 that is shown in FIG. 3A, with various internal organs depicted, including the heart 305, lungs 307 and rib cage boundary 310. In addition, for reference, FIG. 3D illustrates various anatomical designations of the laboratory rat, including left and right pectoral regions, a medial region, a right mid-lateral region, axilla and abdomen.

As depicted in FIG. 3B, the current lead field, which is associated with the current electrodes 253A and 253B, largely overlaps the voltage lead field, which is associated with the voltage electrodes 256A and 256B. Accordingly, both lead fields pass through substantially the same organs and internal tissue; and as further depicted, both lead fields pass through the heart 305 and the lungs 307. Accordingly, a resulting thoracic impedance signal (an example of which is shown in a plot 320 of FIG. 3E) may include both a respiration component 321 and a cardiac component 322.

FIG. 3C illustrates a different lead arrangement, which, in some implementations can yield a cleaner respiration signal. In particular, as shown in FIG. 3C, current electrodes 253A and 253B can be placed laterally and mid-laterally, with the former being placed near the axilla and the latter being placed near the intersection of the rib cage and the abdomen. One voltage electrode 256A can be placed medially in the left or right pectoral region, and the second voltage electrode 256B can be placed mid-laterally on a left ventral side of the thorax.

As shown in this example arrangement of electrodes, the current lead field emanates from the right lateral side of the test subject, passing through the lungs and heart. The voltage lead field primarily passes through the lungs, with a relatively smaller portion passing through the heart than the portion in the electrode configuration shown in FIG. 3B. That is, the current and voltage fields substantially intersect in the right lung (from the test subject's perspective), and the fields do not substantially intersect in the heart. Substantially intersecting in this context can include, for example, intersecting to a large extent. In particular, for example a larger percentage of the intersection of the fields (e.g., 51%, 75%, 95%, etc.) occurs in the lung; in contrast, although the voltage and current fields may interest to a small degree in the heart, the fields do not, in this example, substantially intersect in the heart. Accordingly, the resulting thoracic impedance signal, an example of which is shown in plot 340 of FIG. 3F, has less cardiac component, and may thus be better suited for respiration studies.

Numerous variations are possible and contemplated. For example, in other implementations, the group of electrodes can be positioned in substantially the same places as shown in FIG. 3C, but individual electrodes can be reversed. More particularly, the current signal can be provided by electrodes C and D (e.g., electrodes 253A and 253B), and the voltage can be measured by electrodes A and B (e.g., electrodes 256A and 256B). As another example, the current signal can be provided by electrodes B and D, and the voltage can be measured by electrodes A and C. In such an implementation, the voltage field may be particularly focused on the upper lobe of the right lung. Other variations are possible to orient the lead fields in a manner that is optimally suited for obtaining particular physiological parameters. The above description focuses on obtaining respiration parameters, but lead fields could be arranged such that the electrodes are configured to obtain other physiological parameters, such as cardiac parameters (e.g., in a manner that larger excludes respiration signals).

The same principles described above of focusing lead fields to advantageously obtain particular physiological parameters can be applied to any test subject. In particular, the above descriptions are provided in reference to a laboratory rat, but the same principles can be applied to larger laboratory animals, such as canines, primates, porcine, ovine, bovine, equine, etc. Moreover, the same principles can be human patients.

By positioning electrodes to maximize certain signals and minimize other signals, the signal-to-noise ratio can be improved, and less filtering may be needed. In addition, resulting data may be more accurate and may further be more immune to other common sources of interference. For example, electrical interference in the voltage signal related to muscle movement may have less effect, or be more easily filtered out, if the desired physiological signal (e.g., a respiration signal associated with tidal volume) is maximized through optimal or advantageous electrode placement.

The above examples are provided in the context of a laboratory rat, but the principles described herein can be applied to various other test subjects, including, for example, canines, porcine subjects, ovine subject, bovine subjects, equine subjects, and other animal subjects for which physiological measurements are advantageous. Moreover, the principles described herein can further be applied to human patients.

Whatever electrode configuration is employed, a voltage signal that is obtained can be processed in various ways, many of which are described in detail in application Ser. No. 11/933,872, filed Nov. 1, 2007, by Moon et al. For example, an impedance signal may be determined at the implantable device 108, from the current and voltage signals. Cardiac and respiration components of this signal may be filtered as necessary, and appropriate values may be stored in the device 108 for later retrieval, or transmitted in real time a system external to the implantable portion 108. Alternatively, values representative of the voltage signal (e.g., discrete, time-ordered values) may be stored or transmitted, and impedance may be calculated outside the implantable device. Numerous data processing actions are possible and contemplated.

A number of implementations have been described. Nevertheless, it will be understood that various modifications may be made without departing from the spirit and scope of the disclosed implementations. For example, various examples are provided in the context of testing or research of pharmaceutical compounds. However, the reader will appreciate that the systems and methods described herein can be employed to test respiratory effect (or other physiological effect) of any kind of substance, such as for example, substances related to bio-defense or bio-weaponry, substances associated with environmental concerns; or respiratory effect (or other physiological effect) of any kind of stimulus, such as neuron-stimulation. In addition, the systems and methods described herein can be employed to determine or study disease progression during animal model development, or for any other kind of basic research. The systems and methods can be employed in various types of living beings, for various purposes—including all types and sizes of laboratory animals (e.g., rodents, canines, non-human primates, pigs, etc.), other animals that may be used in basic research (e.g., horses, fish, birds, etc.), as well as in human patients. Accordingly, other implementations are within the scope of the following claims.

What is claimed is:

1. A method of measuring lung impedance of a subject, the method comprising:
positioning current-injection electrodes on or within the subject in a configuration such that a current injected between the current-injection electrodes propagates substantially through a first lung of the subject, and not through a heart and a second lung of the subject;
positioning voltage-measurement electrodes on or within the subject in a configuration such that voltage measuring fields propagate substantially through the first lung and the second lung of the subject, but not through the heart of the subject; and
injecting a current between the current-injection electrodes, as positioned, and measuring a resulting voltage between the voltage-measurement electrodes, as positioned, to obtain an impedance measure across lung tissue.

2. The method of claim 1, wherein the current-injection electrodes comprise a first electrode positioned laterally on a right side of the subject, and a second electrode positioned mid-laterally on the right side of the subject.

3. The method of claim 2, wherein the voltage-measurement electrodes comprise a third electrode positioned in a pectoral region of the subject, and a fourth electrode positioned mid-laterally.

4. The method of claim 3, wherein the current-injection electrodes and the voltage-measurement electrodes are implanted within the subject.

5. The method of claim 3, wherein the current-injection electrodes and the voltage-measurement electrodes are surface electrodes placed on a surface of the subject.

6. The method of claim 3, wherein the first electrode is positioned near an axilla.

7. The method of claim 6, wherein the second electrode is positioned near an intersection of a rib cage and an abdomen of the subject.

8. The method of claim 3, wherein the fourth electrode is positioned on a left front side of a thorax of the subject.

9. The method of claim 1, wherein the current-injection electrodes and the voltage-measurement electrodes are implanted within the subject.

10. The method of claim 1, wherein the current-injection electrodes and the voltage-measurement electrodes are surface electrodes placed on a surface of the subject.

11. The method of claim 1, further comprising injecting current and measuring the resulting voltage multiple times over a time period to monitor respiration of the subject over the time period.

12. A method of measuring a respiration parameter in a living being, the method comprising:
implanting in the living being a system, the system comprising a) a wireless transmitter; b) three distinct lead wires; and c) four distinct electrodes disposed on the three distinct lead wires, wherein each of the three distinct lead wires has disposed thereon at least one of the four distinct electrodes and wherein implanting the three distinct lead wires comprises implanting the three lead wires subcutaneously or sub-muscularly;
injecting a current between two of the four distinct electrodes to create a current field in the living being, and measuring a resulting voltage between the other two of four distinct electrodes;
transmitting from the wireless transmitter to a receiver that is external to the living being a value corresponding to the measured voltage; and
determining from the value a respiration parameter for the living being.

13. The method of claim 12, wherein the living being has an abdomen, a thorax generally bounded by a rib cage, and an axilla; wherein implanting the system comprises implanting the three distinct lead wires such that a) a first of the four electrodes is positioned on a right side of the thorax, mid-laterally near an intersection of the rib cage and the abdomen; b) a second of the four electrodes is positioned on the right side of the thorax near the axilla; c) a third of the four electrodes is implanted medially in a left or right pectoral region; and d) a fourth of the four electrodes is implanted mid-laterally on a left ventral side of the thorax.

14. The method of claim 13, wherein injecting the current comprises injecting the current between the first and second electrodes, and measuring the voltage comprises measuring the voltage between the third and fourth electrodes.

15. The method of claim 13, wherein injecting the current comprises injecting the current between the third and fourth electrodes, and measuring the voltage comprises measuring the voltage between the first and second electrodes.

16. The method of claim 13, wherein implanting the three distinct lead wires comprises implanting the three distinct lead wires such that the third electrode is disposed cranially relative to the fourth electrode.

17. The method of claim 12, wherein the system comprises four distinct lead wires, and wherein one of the four distinct electrodes is disposed on each of the four distinct lead wires.

18. The method of claim 12, wherein implanting the system comprises subcutaneously implanting the three distinct lead wires such that a) the current field extends into a thorax of the living being in a manner that intersects lung structures of the living being, and b) the voltage is measured form a voltage field that intersects the lung structures but does not substantially intersect heart structures of the living being.

19. A method of measuring a respiration parameter in a living being, the method comprising:
implanting in the living being a system, the system comprising a) a wireless transmitter; b) three distinct lead wires; and c) four distinct electrodes disposed on the three distinct lead wires, wherein each of the three distinct lead wires has disposed thereon at least one of the four distinct electrodes and wherein implanting the three distinct lead wires comprises implanting the three lead wires subcutaneously or sub-muscularly;
developing a voltage potential between two of the four distinct electrodes to create a voltage field in the living being, and measuring a resulting current between the other two of four distinct electrodes;
transmitting from the wireless transmitter to a receiver that is external to the living being a value corresponding to the measured current; and
determining from the value a respiration parameter for the living being.

20. A method of measuring lung impedance of a subject, the method comprising:
positioning current-injection electrodes within the subject in a configuration such that a current injected between the current-injection electrodes propagates as a plurality of current fields through at least one lung of the subject;
positioning voltage-measurement electrodes within the subject in a configuration such that a) corresponding voltage-measuring fields intersect the plurality of current fields substantially in the at least one lung and b) the voltage-measuring fields do not substantially intersect the plurality of current fields in a heart of the subject; and
injecting a current between the current-injection electrodes, as positioned, and measuring a resulting voltage between the voltage-measurement electrodes, as positioned, to obtain an impedance measure across tissue of the at least one lung.
